(19) United States
(12) Patent Application Publication
(10) Pub. No.: US 2011/0129905 A1
(43) Pub. Date: Jun. 2, 2011

(54) MODIFIED PICHIA STRAIN

(76) Inventors: Karin Lindkvist, Dalby (SE); Cecilia Geijer, Goteborg (SE); Gerhard Fischer, Goteborg (SE); Urszula Kosinski-Eriksson, Goteborg (SE); Madelene Palmgren, Kungsbacka (SE)

(21) Appl. No.: 13/054,815

(22) PCT Filed: Jul. 15, 2009
(86) PCT No.: PCT/SE2009/000369

§ 371 Date: Jan. 19, 2011

(30) Foreign Application Priority Data
Jul. 21, 2008 (SE) 0801721-2

(51) Int. Cl. C12N 1/16

(52) U.S. Cl. 435/255.5

(57) ABSTRACT

The present invention generally relates to a modified strain of yeast. More specifically, it relates to a strain of Pichia pastoris, deficient in its native yeast aquaporin Aqy1 or expressing non-functional Aqy1, as well as related methods and uses. In a first aspect, the present invention comprises a strain of the organism P. pastoris that is deficient in Aqy1.

### FIELD OF THE INVENTION

The present invention generally relates to a modified strain of Pichia. More specifically, it relates to a strain of Pichia pastoris, deficient in its native yeast aquaporin Aqy1 or expressing non-functional Aqy1, as well as related methods and uses.

### BACKGROUND OF THE INVENTION

Aquaporins are integral membrane proteins that form pores in the membrane of biological cells. Aquaporins selectively transport water in and out of cells, while preventing the passage of ions or other solutes (Fu and Lu, 2007).

Since aquaporins regulate the water balance in all living cells, they are of broad interest in various fields such as medicine, agriculture, biological assays etc. By understanding and being able to control the action of aquaporins, a variety of technical applications are possible. For example, the recent discovery of aquaporin involvement in cell proliferation and migration suggest that they play a key role in tumor biology (Verkman et al., 2008). The precise physiological roles of aquaporins in liver or fat cells are partly unknown but their dramatically altered expression following metabolic disease (e.g. diabetes) points to essential roles in metabolism,(Hibuse et al., 2005). Hence, aquaporins are potential targets for drugs. For instance, blockers of kidney aquaporins will function as diuretics, blockers of brain AQPs would diminish fatal brain swelling (edema) upon stroke or trauma and skin AQP blockers are expected to reduce production of sweat and gland fluid in the gastrointestinal and respiratory tracts (Agre et al., 2002).

Some 13 aquaporins have yet been discovered in mammals, and six of these are located in the kidney. When studying aquaporins, as well as other membrane proteins, it is common to express them in suitable host systems, such as yeast. In particular, P. pastoris is a common host for the expression and production of membrane proteins such as aquaporins (Nyblom et al., 2007). However, P. pastoris itself expresses a native aquaporin, referred to herein as Aqy1.

Most surprisingly, the present inventors have recently discovered that Aqy1 is also produced in large quantities while non-native membrane proteins are expressed in P. pastoris. The Aqy1 acts as a contaminant in crystallization when studying membrane proteins expressed in P. pastoris, in particular when studying other aquaporins. In water permeability assays using spheroplasts isolated from P. pastoris cells, Aqy1 causes an increased water transport background.

While the use of assays for aquaporins using P. pastoris are known in the art, such approaches are not aware of the novel discovery of the Aqy1 contaminant, nor the inventive solution to this problem. The international patent application WO/2004/077010 discloses methods for using aquaporin polypeptides to identify agents that can modulate the activity of these water channels.

### SUMMARY OF THE INVENTION

The present invention provides for a modified strain of P. pastoris, deficient in Aqy1 or expressing non-functional Aqy1. The inventors have found that a human aquaporin, hAQP1, can be overproduced using such a strain, and that P. pastoris spheroplasts deficient in Aqy1 or expressing non-functional Aqy1 yield considerably lower background in water transport assays.

Thus, in a first aspect, the present invention comprises a strain of the organism P. pastoris that is deficient in Aqy1.

In another aspect, the present invention comprises a strain of the organism P. pastoris expressing non-functional Aqy1.

In a further aspect, the invention comprises the use of such an Aqy1-deficient strain or a strain expressing non-functional Aqy1 as a host for the expression of proteins, including—but not limited to—membrane proteins, soluble proteins and excreted proteins.

In still a further aspect, the invention comprises the use of such an Aqy1-deficient strain or a strain expressing non-functional Aqy1 as a host for the expression of a membrane protein, in particular an aquaporin.

In yet another aspect, the invention comprises the use of an Aqy1-deficient strain or a strain expressing non-functional Aqy1 of Pichia pastoris in cell based functional assays, in particular such assays wherein water and/or solute transport through an aquaporin is measured, and most preferably where it is measured to determine blockers and inhibitors of aquaporins.

In yet another aspect, the invention comprises such uses in cell based functional assays, wherein the functional assay makes use of the shrinkage, swelling or bursting of the cells, where the shrinkage, swelling or bursting is caused by an osmolar gradient, and/or where the cell volume change is measured by light scattering. In a further aspect, the invention comprises such uses where a stopped-flow machine is also used.

### DETAILED DESCRIPTION OF THE INVENTION

### Definitions

As used herein, the terms “Aqy1 deficient strain” or “deficient in Aqy1” etc. signify any modified strain of Pichia pastoris that does not express the native aquaporin Aqy1 or express a non-functional Aqy1. As a person skilled in the art will appreciate, there are several means available to achieve this inventive concept, and the invention should not be construed as limited to the specific examples or enabling disclosure herein.

As used herein, the terms “cell based functional assay” or “cell based assay” should be construed in the broadest sense, and could for example comprise, without limitation, aquaporin assays to screen for inhibitors or verify functionality of new substances without unwanted background disturbance from Aqy1. The term comprises live cell assays, including spheroplast assays, patch clamping, and related whole cell assays that are known in the art.

### Use and Practice of the Invention

The novel P. pastoris strain has many uses, as is also detailed in the claims. The Aqy1-deficient strain is ideal for studying the structural biology of membrane proteins, since the invention solves the problem of Aqy1 co-purifying, accidentally crystallizing “false leads”, which would reduce the chances of recovering crystals of the target protein. The structural biology of membrane proteins is of great interest in drug design, and a further application in this regard would be co-crystallisation with potential inhibitor compounds or chemical fragments.

The novel strain can also be used as a source for assays. This includes cell based assays as defined above, or using a protein produced in the inventive strain. The purified protein product, uncontaminated by native Aqy1, could be used for liposome, light scattering or NMR-binding assays, as well as for isothermal calorimetry (ITC) or other biophysical methods.

The novel strain can also be used for protein production in general, with the advantage of avoiding the Aqy1 contaminant and possibly also achieving better expression levels of the target protein as a result. The strain is particularly suited to express membrane proteins in this manner, especially aquaporins, ion channels, GPCRs etc.

### EXAMPLES

The following examples are provided for illustrative purposes only, and shall not be construed as limiting the scope of the present invention.

### Example 1

### Disruption of AQY1

To disrupt AQY1 in Pichia pastoris, the GS115 (his4) strain background (Invitrogen) was used.

We created a pPICZB-vector (Invitrogen) containing AQY1 by extracting the genome from Pichia pastoris and amplifying the AQY1 gene with PCR using primers A and B. The anti-sense primer fused the gene with a his6-tag. Furthermore, EcoRI- and XbaI-restriction sites were added which were used to ligate the gene into the pPICZB-vector, thus yielding the pPICZB-his-construct.

Primer A:
5′-TGCAGAATTCAAAATGCCTGACATTGAAAACC-3′
Primer B:
5′-GTACTCTAGATCAGTGATGGTGGTGATGGTGAGCATCTGAATCTTG
GC-3′

The AQY1 gene in this construct was disrupted by inserting Saccharomyces cerevisiae HIS4 with its own promoter. The HIS4 promoter and the open reading frame (ORF) was amplified by PCR, using primers C and D designed to add PstI restriction sites at the ends of the produced DNA. pPICZB-AQY1-his was linearized by digestion using PstI cutting in the ORF of AQY1. The linearized vector and the PstI digested PCR product of HIS4 were ligated and transformed into E. coli and transformants were selected for on Zeocin containing plates. Successful insertion of HIS4 into the ORF of AQY1 in the pPICZB vector was confirmed by PCR (primers E and F). This vector was then used as template in a PCR reaction to amplify the AQY1-HIS4-AQY1 construct (primers F and G). The PCR product was precipitated and subsequently transformed into P. pastoris GS115 using standard protocols. Transformants were selected for histidine prototrophy and verification of the disruption of AQY1 was confirmed by PCR and phenotypic analysis (cf. below).

| | |
|---|---|
| Primer C: | 5′-TAGACTGACTGCAGACAATCCTGACAACCAGCA-3′ |
| Primer D: | 5′-AGCCAGTTCTGCAGCGTTAGTGTTCGGTTTCCA-3′ |
| Primer E: | 5′-TCTCGACGTCCTTCAATGA-3′ |
| Primer F: | 5′-ATGCCTGACATTGAAAACC-3′ |
| Primer G: | 5′-AGCATCTGAATCTTGGCCT-3′ |

### Example 2

### Freezing and Thawing to Confirm Disruption

Cells were grown in 10 ml BMGY at 30° C. for 24 hours, 10 OD600 units were then transferred to 10 ml BMMY for final OD600=1 and incubated for 24 hours at 30° C.

For the freezing and thawing assay, cells were transferred to 1 ml YPD in Eppendorf tubes, final OD₆₀₀=0.0005. An aliquot of 50 ul was plated on YPD plates before stress to serve as reference (100% survival). In total 8 cycles of freezing in liquid nitrogen for 30 seconds and thawing for 3 minutes in water bath at 30° C. followed by plating of aliquots on YPD plates were carried out. Survival was scored by counting colony forming units (CFUs).

TABLE 1
CFUs per cycles of freezing and thawing

| | Strain/cycles | | | | |
|---|---|---|---|---|---|
| | 0 | 2 | 4 | 6 | 8 |
| X-33 wild type | 2105 | 1529 | 1148 | 872 | 511 |
| aqy1 deletion | 1981 | 1395 | 334 | 133 | 42 |

It has previously been shown that wildtype strains can withstand the stress of freezing and thawing better than aqua-porin deletion strains (Tanghe et al., 2002). The reduced number of CFUs in the aqy1 deletion strain proves that AQY1 was successfully disrupted.

### Example 3

### Spheroplast Assay to Confirm Reduction of Water Transport in Aqy1 Deletion Strain

Pichia pastoris cells, both wildtype X33 cells and the aqy1 deletion strain, were grown for 24 h in 10 ml BMGY in a rotary shaker at 220 rpm and 30° C. Subsequently, the protein production was induced by growing the cells in 10 ml BMMY for 24 h as above, starting at OD₆₀₀=1 by spinning down the respective amount at 3000 xg, 10 min, 20° C.

The cells were sedimented at 3000 xg for five minutes at 20° C. and resuspended in 1.4 ml/g wet cell weight of TE-buffer (100 mM Tris, pH 8.0, 100 mM EDTA) and diluted to a final volume of 3.5 ml/g wet cell weight with dH₂O. 2-mercapto-ethanol (( 1/200th of volume)/wet cell weight) was added and incubation took place in a rotary shaker at 220 rpm, 30° C. for 45 minutes, whereupon the cells were washed two times by sedimentation as above and resuspension in 10 ml S-buffer (1.2 M Sorbitol, 10 mM MES pH 6.0). Subsequently, the cells were resuspended in 10 ml S-buffer supplemented with Zymolyase 20T (Seikagaku Corp, 50 U/g wet cell weight) and incubated in a rotary shaker at 150 rpm, 30° C. for 30 minutes. The appearance of the spheroplasts was monitored under a microscope, removal of the cell wall was verified by the observation of bursting upon the addition of dH2O (1:1) due to hypo-osmotic shock. The cells were washed three times by sedimenting at 2000 xg for 5 min at 4° C. intermittently. Finally, the spheroplasts were resuspended in 10 ml S-buffer.

Change in light scattering at 440 nm was observed upon mixing with equal amounts of 1.8 M Sorbitol Buffer in a stopped-flow spectrophotometer device (Biologic Science Instruments). Water transport activity measurements using stopped-flow spectrometry from P. pastoris spheroplasts with the aqy1 gene disrupted compared to the wildtype strain (with endogenous Aqy1) gave k-values of 0.35 and 2.6, respectively. Hence the intrinsic water transport rate of P. pastoris has been lowered 7.5 times upon the disruption of the aqy1 gene.

### REFERENCES

Agre, P., King, L. S., Yasui, M., Guggino, W. B., Ottersen, O. P., Fujiyoshi, Y., Engel, A. and Nielsen, S. (2002) Aquaporin water channels—from atomic structure to clinical medicine. J Physiol, 542, 3-16.
Fu, D. and Lu, M. (2007) The structural basis of water permeation and proton exclusion in aquaporins. Mol Membr Biol, 24, 366-374.
Hibuse, T., Maeda, N., Funahashi, T., Yamamoto, K., Nagasawa, A., Mizunoya, W., Kishida, K., Inoue, K., Kuriyama, H., Nakamura, T., Fushiki, T., Kihara, S. and Shimomura, I. (2005) Aquaporin 7 deficiency is associated with development of obesity through activation of adipose glycerol kinase. Proc Natl Acad Sci USA, 102, 10993-10998.
Nyblom, M., Oberg, F., Lindkvist-Petersson, K., Hallgren, K.,

Findlay, H., Wikstrom, J., Karlsson, A., Hansson, O., Booth, P. J., Bill, R. M., Neutze, R. and Hedfalk, K. (2007) Exceptional overproduction of a functional human membrane protein. Protein Expr Purif, 56, 110-120.
Tanghe, A., Van Dijck, P., Dumortier, F., Teunissen, A., Hohmann, S. and Thevelein, J. M. (2002) Aquaporin expression correlates with freeze tolerance in baker's yeast, and overexpression improves freeze tolerance in industrial strains. Appl Environ Microbiol, 68, 5981-5989.
Verkman, A. S., Hara-Chikuma, M. and Papadopoulos, M. C. (2008) Aquaporins-new players in cancer biology. J Mol Med, 86, 523-529.
Fischer G, Kosinska-Eriksson U, Aponte-Santamaría C, Palmgren M, Geijer C, Hedfalk K, Hohmann S, de Groot B L, Neutze R, Lindkvist-Petersson K (2009) Crystal structure of a yeast aquaporin at 1.15 angstrom reveals a novel gating mechanism. PLoS Biol, 7(6)

1. A strain of the organism Pichia pastoris, characterized in that it is deficient in Aqy1.

2. A strain of the organism Pichia pastoris, characterized in expressing non-functional Aqy1.

3. Use of the strain according to claim 1 as a host for the expression of proteins, including—but not limited to—membrane proteins, soluble proteins and excreted proteins.

4. Use of the strain according to claim 1 as a host for the expression of a membrane protein.

5. Use according to claim 3, wherein the membrane protein is an aquaporin.

6. Use of the strain according to claim 1 in cell based functional assays.

7. Use according to claim 6 wherein water and/or solute transport through an aquaporin is measured.

8. Use according to claim 7, wherein a decrease in water and/or solute transport is measured to determine blockers and inhibitors of aquaporins.

9. Use of the strain according to claim 6, wherein the functional assay makes use of the swelling or shrinkage of the cells.

10. Use according to claim 9, wherein the swelling or shrinkage is caused by an osmolar gradient.

11. Use according to claim 9, wherein the swelling or shrinkage is measured by light scattering.

12. Use according to claim 11, wherein a stopped-flow machine is used.
